SEQUENCE LISTING (8 sequences)

SEQ ID NO: 1 - DNA, 30 bp, Artificial Sequence
  Primer P1
atagatatcatgctgctgctgctgctgctg

SEQ ID NO: 2 - DNA, 32 bp, Artificial Sequence
  Primer P2
atagcggccgccccgactctagagtaacccgg

SEQ ID NO: 3 - DNA, 4677 bp, Artificial Sequence
  Expreesion vector containing cDNA of SEAP
ggtaccgagctcttacgcgtgctagcccgggctcgagatctgcgatctaagtaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 4 - DNA, 36 bp, Artificial Sequence
  Primer P3
atagcggccgctgagagccaggaccaaagctcctta

SEQ ID NO: 5 - DNA, 33 bp, Artificial Sequence
  Primer P4
atagaattcttagtttgaagggcattcgcactt

SEQ ID NO: 6 - DNA, 1089 bp, Homo sapiens
  cDNA SELENOP
gagagccaggaccaaagctccttatgtaagcaacccccagcctggagcataagagatcaagatccaatgctaaactccaatggttcagtgactgtggttgctcttcttcaagccagctgctacctgtgcatactgcaggcatctaaattagaagacctgcgagtaaaactgaagaaagaaggatattctaatatttcttatattgttgttaatcatcaaggaatctcttctcgattaaaatacacacatcttaagaataaggtttcagagcatattcctgtttatcaacaagaagaaaaccaaacagatgtctggactcttttaaatggaagcaaagatgacttcctcatatatgatagatgtggccgtcttgtatatcatcttggtttgcctttttccttcctaactttcccatatgtagaagaagccattaagattgcttactgtgaaaagaaatgtggaaactgctctctcacgactctcaaagatgaagacttttgtaaacgtgtatctttggctactgtggataaaacagttgaaactccatcgcctcattaccatcatgagcatcatcacaatcatggacatcagcaccttggcagcagtgagctttcagagaatcagcaaccaggagcaccaaatgctcctactcatcctgctcctccaggccttcatcaccaccataagcacaagggtcagcataggcagggtcacccagagaaccgagatatgccagcaagtgaagatttacaagatttacaaaagaagctctgtcgaaagagatgtataaatcaattactctgtaaattgcccacagattcagagttggctcctaggagctgctgctgccattgtcgacatctgatatttgaaaaaacagggtctgcaatcacctgccagtgtaaagaaaacctcccatctttatgtagctgccagggacttcgggcagaggagaacataactgaatcttgtcagtgccgtttgcctccagctgcctgccaaataagtcagcagcttatacccacagaagccagtgccagttgccgctgcaagaatcaggcaaaaaagtgcgaatgcccttcaaactaa

SEQ ID NO: 7 - DNA, 2631 bp, Artificial Sequence
  cDNA of fusion protein SEAP-SELENOP
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccgctgagagccaggaccaaagctccttatgtaagcaacccccagcctggagcataagagatcaagatccaatgctaaactccaatggttcagtgactgtggttgctcttcttcaagccagctgctacctgtgcatactgcaggcatctaaattagaagacctgcgagtaaaactgaagaaagaaggatattctaatatttcttatattgttgttaatcatcaaggaatctcttctcgattaaaatacacacatcttaagaataaggtttcagagcatattcctgtttatcaacaagaagaaaaccaaacagatgtctggactcttttaaatggaagcaaagatgacttcctcatatatgatagatgtggccgtcttgtatatcatcttggtttgcctttttccttcctaactttcccatatgtagaagaagccattaagattgcttactgtgaaaagaaatgtggaaactgctctctcacgactctcaaagatgaagacttttgtaaacgtgtatctttggctactgtggataaaacagttgaaactccatcgcctcattaccatcatgagcatcatcacaatcatggacatcagcaccttggcagcagtgagctttcagagaatcagcaaccaggagcaccaaatgctcctactcatcctgctcctccaggccttcatcaccaccataagcacaagggtcagcataggcagggtcacccagagaaccgagatatgccagcaagtgaagatttacaagatttacaaaagaagctctgtcgaaagagatgtataaatcaattactctgtaaattgcccacagattcagagttggctcctaggagctgctgctgccattgtcgacatctgatatttgaaaaaacagggtctgcaatcacctgccagtgtaaagaaaacctcccatctttatgtagctgccagggacttcgggcagaggagaacataactgaatcttgtcagtgccgtttgcctccagctgcctgccaaataagtcagcagcttatacccacagaagccagtgccagttgccgctgcaagaatcaggcaaaaaagtgcgaatgcccttcaaactaa

SEQ ID NO: 8 - protein, 876 aa, Artificial Sequence
  Fusion Protein SEAP-SELENOP
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGYSRVGAAAESQDQSSLCKQPPAWSIRDQDPMLNSNGSVTVVALLQASCYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSCCCHCRHLIFEKTGSAITCQCKENLPSLCSCQGLRAEENITESCQCRLPPAACQISQQLIPTEASASCRCKNQAKKCECPSN

(19) United States
(12) Patent Application Publication
(10) Pub. No.: US 2024/0310387 A1
(43) Pub. Date: Sep. 19, 2024

(54) COMPOSITE BIOMARKER FOR THE IDENTIFICATION OF SELENIUM DEFICIENCY IN A BODILY FLUID

(71) Applicant: BERYSOL GMBH, Berlin (DE)

(72) Inventor: Lutz SCHOMBURG, Berlin (DE)

(73) Assignee: BERYSOL GMBH, Berlin (DE)

(21) Appl. No.: 18/575,507

(22) PCT Filed: Jun. 28, 2022
(86) PCT No.: PCT/EP2022/067808

§ 371 Date: Dec. 29, 2023

(30) Foreign Application Priority Data
Jun. 29, 2021 (DE) 10 2021 206 749.2
Aug. 19, 2021 (DE) 10 2021 209 147.4

(51) Int. Cl. G01N 33/68; G01N 33/53

(52) U.S. Cl. CPC G01N 33/6893; G01N 33/5308; G01N 2800/50

(57) ABSTRACT

Instant invention discloses a method for evaluating the risk of selenium (Se) deficiency in a subject by determining both the amount of selenium or selenoprotein P and the amount of antibodies against selenoprotein P, estimating or calculating a risk index score for the Se deficiency of said subject using the obtained values, and the use of the method of the invention or of the risk index score obtained according to the invention in the monitoring of Se supplementation, especially in subjects suffering from a disease related to a selenium deficit.

Instant invention discloses a method for evaluating the risk of selenium (Se) deficiency in a subject by determining both the amount of Se or selenoprotein P and the amount of antibodies against selenoprotein P, optionally calculating a risk index score for the Se deficiency of said subject using the obtained values, and the use of the method of the invention or of the risk index score obtained according to the invention in the monitoring of Se supplementation, especially in subjects suffering from a disease related to a Se deficit.

Selenoprotein P (abbreviations Sepp1, SeP, SELP, SePP, SELENOP, hereinafter “SELENOP”) is a plasma selenoprotein, which is known to serve as marker of selenium (Se) status and deficiency [1]. SELENOP serum or plasma concentration reaches a saturation level used as indicator of a sufficiently high, i.e., replete and health supporting Se intake [2,3]. Thus, the determination of SELENOP has become an important tool for the assessment of the nutritional Se requirement [4-6].

Structurally, human SELENOP is a protein containing 381 amino acid residues of which ten are predicted to be selenocysteine (Sec) residues at positions 59, 300, 318, 330, 345, 352, 367, 369, 376 and 378. However, the precise number of Sec per SELENOP is variable, and at some predicted Sec positions other residues are found, causing on average a considerably lower Se content per SELENOP molecule than predicted (mean±SD: 5.4±0.5) [7]. Its secreted form (after cleavage of the signal sequence) contains 362 amino acid residues and may contain post-translational modifications, which can include phosphorylation and multiple sites of glycosylation [8,9]. Moreover, several variants including fragments containing the N- or C-terminal part of SELENOP have been described [8, 10, 11].

Circulating concentrations of SELENOP have been shown to be saturable, i.e., SELENOP is supposed to reach a maximal concentration in the circulation of 5-7 mg/l under normal conditions [2,12]. SELENOP is therefore used as a biomarker for Se deficiency and for the determination of Se requirement. Se supplementation studies have indicated that SELENOP serum or plasma concentration is an easily accessible marker of human Se nutritional status, whereas quantification of the trace element concentration of Se in the blood requires higher efforts due to the need of physico-chemical/spectroscopic techniques (e.g., atom absorption spectrometry, X-ray fluorescence or mass spectrometry) [2,5,13-17].

Populations and subjects with sub-optimal expression levels and circulating concentrations of less than 5 mg/l SELENOP are considered and classified as Se-deficient, i.e., in need of supplemental Se administration. Once, the Se serum concentration is replete, i.e., in the range of at least 120-130 μg Se/l, circulating SELENOP becomes saturated and it has been reported that SELENOP concentration does not increase above 5-7 mg/l, even upon further Se supplementation [2,5,15]. The respective subject with 5-7 mg/l of SELENOP is considered as Se-replete. It was believed that higher intake levels of Se are not affecting the circulating SELENOP concentrations any further, and that SELENOP concentrations above 5-7 mg/l may represent a rare status, resulting from a specific genotype, disease or other individual reasons, but are not caused by high Se intake alone. Very high SELENOP concentrations have been described in relation to obesity or pulmonary arterial hypertension [19], but not in response to supplementation with Se-containing substances. Elevated circulating SELENOP levels have been reported in patients with type 2 diabetes and pre-diabetic conditions and were shown to be related to atherosclerosis [20]. In contrast, SELENOP level concentration is decreased in sepsis and is presumably the cause of the decline in Se level or a decreased release of the trace element by the liver [22]. Significantly decreased circulating SELENOP levels that were associated to the metabolic syndrome status were also found in patients with documented cardio-vascular diseases [23].

Insufficient supply with the trace element Se causes a Se deficit that is associated with reduced Se and selenoprotein (especially SELENOP) concentrations in bodily fluids, which constitutes a risk factor for many diseases and disease-related complications. A Se deficit resulting in a low Se status is associated with disease risks, e.g., Hashimoto's thyroiditis and Graves' disease. On the other hand, cases of Se intoxication have been analyzed and serum Se concentrations above the reference values have been reported (e.g., paradise nut paradox) causing eventually hair loss as well as fingernail and toenail weakness and loss [24, 25].

Currently, there are several approaches to determine the Se status of an individual. It can be determined by the measurement of the concentration, resp., amount of Se or of the protein biomarker SELENOP in a bodily fluid of an individual [2, 26].

SELENOP concentrations appear to correlate with the Se concentrations in bodily fluids (e.g., blood, serum, plasma, mother milk, cerebrospinal fluid) over a broad concentration range qualifying SELENOP as a suitable biomarker of the Se status. Se supplementation studies [2,27,28] suggest that SELENOP serum or plasma level is a preferred biomarker of Se status in humans. In these clinical studies, a highly significant correlation was found between serum Se and SELENOP levels [2,27,28]. Likewise, SELENOP serves as Se deficiency biomarker, because it was shown that the SELENOP plasma level decreases as the grade of a deficient Se status increases [29-31].

Currently, the understanding of Se metabolism and especially the role of a Se deficit in development and cure of many pathological conditions is rapidly growing.

Due to the beneficial health effect of Se, which is mediated through selenoproteins, it has been proposed to improve the Se status and reduce health risks by supplying sufficient amounts of Se and preventing Se from becoming the limiting factor in selenoprotein synthesis. Improvements of the Se status can be achieved by changing the dietary pattern towards food with higher Se content, resp., by administrating Se supplementation [32].

It has also been reported that Se deficit may constitute an important (risk) factor in the generation or amelioration of many diseases, resp., disease-related complications, including, but not limited to: lung cancer, breast cancer, prostate cancer, liver cancer, colon or colorectal cancer, cardio-vascular diseases (arterial hypertension, myocardial infarction, stroke, reperfusion injury), infertility, bacterial infection, sepsis, (poly)trauma, systemic inflammatory response syndrome (SIRS), viral infection (especially influenza A, hepatitis B, hepatitis C, COVID-19), and neurological dysfunctions including early neurodegeneration and seizures, e.g., Alzheimer's disease, Parkinson's disease, and epileptic seizure. For example, it has been shown that the detection of SELENOP can be used to assess the risk in a healthy subject for getting a first cardiovascular event or the assessment of the risk for cardiovascular mortality (WO2019081504A1) or the risk of a subject for getting a heart failure event (WO2020128073A1).

A number of clinical trials have studied the health effects of supplemental Se intake on certain medical aspects, e.g., reduction of disease risk, ameliorating disease symptoms or contributing to therapy when given as a therapeutic adjuvant. The results of different trials with similar groups of subjects have often been controversial. Some major fields in supplemental Se intake trials may serve as an example: The Nutritional Prevention of Cancer (NPC) trial in the USA reported an almost 2-fold reduced prostate cancer incidence rate in subjects taking 200 μg of supplemental Se per day over an average time course of 4.5 years. In contrast the Selenium and Vitamin E Cancer Prevention Study (SELECT) conducted a very similar intervention trial, albeit without reporting positive effects of 200 μg supplemental Se per day on prostate cancer incidence. Similarly, comparable Se supplementation studies in autoimmune thyroiditis have reported conflicting results [33]. The same applies to the effects on mortality of severely diseased patients in the ICU who regularly develop very pronounced Se deficiency. Similar intervention studies with supplemental Se reported conflicting results, i.e., very strong mortality risk reduction versus no significant effects. Unfortunately, the results from investigations wherein Se was supplemented to reduce health risks or improve therapeutic success to date have not always been consistent.

During prophylaxis and therapy of a Se deficit the effects of Se supplementation can be monitored via the determination of Se or SELENOP concentration in a biological sample of a subject. While it is reported that SELENOP concentration reaches a plateau, once a sufficiently high Se status is achieved by Se supply, DE102020002289 A1 teaches that SELENOP concentration may be able to exceed plateau levels, thus also be suitable as a biomarker for Se intoxication.

While a detection method for autoantibodies (aAb) against SELENOP was described and the existence of anti-SELENOP-aAb has been reported in autoimmune thyroid patients [34], the crucial role of anti-SELENOP-aAb (and of anti-SELENOP-Ab in general) in the assessment of the Se status has not been recognized.

Several SELENOP quantification methods are known, such as radioimmunoassay [26,35,36], enzyme-linked immunosorbent assays (ELISA) [37], chemiluminescence immunoassay and sandwich SELENOP-ELISA [38]. Antibody-based sandwich ELISA, preferentially with monoclonal antibodies, may be the most reliable, wide-spread and frequently used technique [7, 21, 26, 39, 40].

Furthermore, WO2020128073A1 teaches that certain SELENOP fragments beside full length SELENOP forms (with signal sequence, secreted or processed form) are particularly suitable in the determination of the amount of SELENOP (cf. Sequence ID No's 1 to 15 of WO2020128073A1), especially in a bodily fluid of an individual.

Commercially available ELISA kits for the detection of SELENOP are available, e.g., Human Selenoprotein P (SELENOP) ELISA kit, Product Code: STE from selenOmed GmbH, Berlin, Germany; Chicken Selenoprotein P (SELENOP1) ELISA Kit, Cat. No. AE56582CH from Wuhan Abebioscience Co., Ltd., Wuhan City, China; Rabbit Selenoprotein P (SELENOP1) ELISA Kit, Cat. No. AE56581RB from Wuhan Abebioscience Co., Ltd.

Thus, SELENOP is particularly known as a suitable marker for monitoring the Se metabolism in Se deficient subjects, i.e., the dynamic progression of the Se status after Se intake of a subject towards a replete Se status, and the decline thereafter.

Thus, it is one of the underlying problems of instant invention to provide a cheap, fast, and reliable determination of the Se status in a subject, to allow appropriate control and monitoring of the required amount of Se to be administrated during supplementation therapy for said subject in need thereof, especially of the assessment of a deficit in the Se status. Such improved identification of the Se status would further allow an appropriate prophylactic and/or therapeutic Se supplementation regimen in a subject, especially to avoid or treat disorders or dysfunctions, which are related to a Se deficit. It is self-evident that the subject's need in Se must be correctly identified during a Se supplementation therapy to avoid worsening of the subject's health status and a potential Se intoxication as well.

It is another underlying problem of the present invention to allow the diagnostic use of a Se status marker for monitoring Se supplementation, especially therapeutic or nutritional supplementation. This is surprisingly achieved by correlating the Se concentration and/or the protein/peptide concentration of SELENOP, i.e., SELENOP or its orthologues in animals in the bodily fluid of an individual together with the relative amount of anti-SELENOP antibodies, especially of anti-SELENOP autoantibodies or therapeutic anti-SELENOP antibodies.

Surprisingly, it was found that it is necessary for the determination of the Se status of an individual not only to identify the absolute or relative amount of observed SELENOP, but also to identify the absolute or relative amount of anti-SELENOP antibodies, particularly of anti-SELENOP autoantibodies, to correctly assess the Se status of an individual. Even if it is not possible to identify the absolute amount of anti-SELENOP autoantibodies without greater effort, an evaluation method was found, which is easy to perform and which allows a fast and reliable assessment of the subject's need for a Se supplementation therapy.

Instant invention overcomes the technical prejudice that a normal value of the Se or SELENOP amount as measurable in the body fluid of a subject would be sufficient to provide the relevant information about the need of Se supplementation of a subject by introducing with a measured value of the amount of anti-SELENOP antibodies a highly relevant additional factor for the assessment of the subject's need of Se supplementation. As taught by instant invention, a Se deficit is possible when anti-SELENOP antibodies are present even if the amount or level of Se or SELENOP as measured by the standard techniques suggest a normal or sufficient level of the Se status.

Accordingly, the methods of instant invention are suitable as a companion diagnostic in the prophylaxis and treatment of a subject suffering from Se deficiency and/or categorized as Se deficient.

Therefore, a first object of instant invention is a method for evaluating the health risk of selenium (Se) deficiency in a subject comprising a) determining in a sample of said subject a measured biological value selected from a1) selenium (Se) and/or a2) selenoprotein P (SELENOP); and b) determining in a sample of said subject a measured biological value of anti-SELENOP-antibodies, preferably aAb(SELENOP).

Alternatively, an object of instant invention is a method for evaluating the health risk associated with selenium (Se) deficiency in a subject comprising a) determining in a sample of said subject a measured biological value selected from a1) selenium (Se) and/or a2) selenoprotein P (SELENOP); and b) determining in a sample of said subject a measured biological value of anti-SELENOP-antibodies, preferably aAb(SELENOP).

In a preferred embodiment of the method of the invention for evaluating the health risk associated with selenium (Se) deficiency or evaluating the risk of selenium (Se) deficiency in a subject the method further comprises the steps c) setting two or more biological parameters into correlation, wherein said biological parameters are represented by the measured biological values obtainable or obtained as defined in steps a) and b); and d) calculating a risk index score for the Se deficiency of said subject using the measured biological values obtainable or obtained as defined i) in step a) and b) or ii) in step c).

In alternative embodiments the sample of said subject for the performance of steps a1, a2 and/or b) is identical or different, preferably identical.

In another embodiment of the method of the invention for evaluating the risk of selenium (Se) deficiency in a subject the risk index score for Se deficiency of said subject is correlated with one or more threshold values, preferably as outlined in Table 1A and 1B.

Another object of instant invention is a method for evaluating the health risk of selenium (Se) deficiency in a subject comprising
a) determining in a sample of said subject a measured biological value selected from
a1) selenium (Se) and/or a2) selenoprotein P (SELENOP); and
b) determining in a sample of said subject a measured biological value of antibodies against SELENOP (SELENOP-Ab); and
c) evaluating the risk of Se deficiency on the basis of the values obtainable as defined in step a and b.

Another object of instant invention is a method for evaluating the health risk of selenium (Se) deficiency in a subject comprising
a) determining in a sample of said subject a measured biological value selected from
a1) selenium (Se) and/or a2) selenoprotein P (SELENOP); and
b) determining in a sample of said subject a measured biological value of antibodies against SELENOP (SELENOP-Ab);
c) setting two or more biological parameters into correlation by using mathematical operators, preferably addition, multiplication, and/or potentiation, wherein said biological parameters are represented by the said measured biological values obtainable as defined in steps a) and b);
d) evaluating the risk of Se deficiency by calculating a risk index score for the Se deficiency of said subject using the values obtainable as defined i) in step a) and b) of claim 1 or ii) in step c).

Another object of instant invention is an in vitro method for the diagnosis of selenium (Se) deficiency in a subject comprising
a) determining in a sample of said subject a measured biological value selected from
a1) selenium (Se) and/or a2) selenoprotein P (SELENOP); and
b) determining in a sample of said subject a measured biological value of antibodies against SELENOP (SELENOP-Ab);
c) setting two or more biological parameters into correlation by using mathematical operators, preferably addition, multiplication, and/or potentiation, wherein said biological parameters are represented by the said measured biological values obtainable as defined in steps a) and b);
d) evaluating the grade of Se deficiency by calculating a risk index score for the Se deficiency of said subject using the values obtainable as defined i) in step a) and b) of claim 1 or ii) in step c).

The term “subject” according to instant invention means a living human or non-human organism, preferably a vertebrate, especially a mammal, more preferably a human. Most preferred, the subject is suffering or is suspected to suffer from a disease related to a selenium deficit. The term “subject” according to other embodiments of instant invention means animals such as a mammal, bird, and/or fish, preferably a companion animal, especially chicken, duck, goose, pig, cattle, goat, sheep, game, horse, camel, cat, dog, rabbit, trout, salmon.

The term “disease related to a selenium deficit” (hereinafter “DRSD”) according to specific, optionally individual, embodiments of instant invention preferably means one or more disease, disorder or dysfunction selected from: sepsis; and/or inflammation; and/or SIRS (systemic inflammatory response syndrome); and/or inflammatory bowel diseases, especially Morbus Crohn and/or colitis ulcerosa; and/or bacterial infection; and/or viral infection, especially an infection causing influenza A, hepatitis B, hepatitis C, or COVID-19; and/or cardio-vascular diseases (CVD) including atherosclerosis and cardiovascular event, especially stroke, myocardial infarction, cardiomyopathy, and/or acute heart failure; and/or cancer, especially breast cancer and/or prostate cancer and/or colorectal cancer and/or lung cancer and/or liver cancer, particularly breast cancer; and/or (poly)trauma; and/or thyroid dysfunction, especially Hashimoto's thyroiditis, Graves' ophthalmopathy; and/or diabetes mellitus type I and/or II; and/or a severe illness treated in an intensive care unit (ICU); and/or severe burn or scald injury; and/or poisoning and/or neurodegenerative diseases, especially epileptic seizure and/or Alzheimer's disease and/or Parkinson's disease and/or Huntington's disease and/or progressive or chronical multiple sclerosis (MS) and/or amyotrophic lateral sclerosis (ALS) and/or infertility and/or hypertension, said aforementioned diseases, disorders or dysfunctions optionally exhibiting clinical symptoms.

Accordingly, in the following separate embodiments of the first object of instant invention the method is evaluating the risk of selenium (Se) deficiency in a subject suffering from one or more of a DRSD, particularly i) sepsis, ii) inflammation, iii) SIRS, iv) inflammatory bowel diseases, v) viral infection, especially influenza A, hepatitis B, hepatitis C, or COVID-19, vi) cardio-vascular diseases (CVD), vii) atherosclerosis, viii) cardiovascular event, ix) stroke, x) myocardial infarction, xi) cardiomyopathy, xii) cancer, xiii) breast cancer, xiv) prostate cancer, xv) colorectal cancer, xvi) lung cancer, xvii) liver cancer, xviii) (poly)trauma, xix) thyroid dysfunction, especially Hashimoto's thyroiditis and Graves' ophthalmopathy, xx) diabetes mellitus type I and/or II, xxi) a severe illness treated in an intensive care unit (ICU), xxii) severe burn or scald injury, xxiii) poisoning, especially with toxic metals like cadmium, mercury or lead, xxiv) a neurodegenerative disease, xxv) epileptic seizure, xxvi) Alzheimer's disease, xxvii) Parkinson's disease, xxviii) Huntington's disease, xxix) progressive or chronical multiple sclerosis (MS), xxx) amyotrophic lateral sclerosis (ALS), xxxi) infertility, or xxxii) hypertension, and in specific embodiments Hashimoto's disease, Graves' ophtalmopathy, influenza A, hepatitis B, hepatitis C, or COVID-19.

The term “sample” according to instant invention and referring to said subject, preferably means a bodily fluid of said subject, more preferred a bodily fluid selected from the group consisting of whole blood, serum, plasma, urine, cerebrospinal liquid (CSF), and saliva. In a preferred embodiment the “sample” as outlined before is selected from the group consisting of whole blood, plasma, and serum.

The term “measured biological value” according to instant invention means preferably any detectable signal representing the relative or absolute amount or concentration of a biological analyte of interest or a respective biomarker thereof in the sample of the subject of investigation in accordance with the suitable detection methods as known by the skilled person. According to instant invention the term “measured biological value” encompasses particularly the relevant biological indicators for the determination of the Se status, which comprise:
a1) trace element selenium (Se); and/or
a2) selenoprotein P (SELENOP); and
b) antibodies against SELENOP (Ab(SELENOP)), preferably aAb(SELENOP), preferably from one identical sample of said subject.

In preferred embodiments of each object of instant invention antibodies against SELENOP (Ab(SELENOP)) are of the autoantibody type (aAb(SELENOP).

In further embodiments of each object of instant invention, the term “anti-SELENOP-antibodies” encompasses, resp., means anti-SELENOP-autoantibodies (aAb(SELENOP)) and/or therapeutic Ab(SELENOP).

In one embodiment of instant invention, the measured biological value of a biological analyte, which is an antibody is represented by the binding index of such antibody. In another embodiment of instant invention, the measured biological value of a biological analyte, which is an antibody against SELENOP, preferably an autoantibody, is represented by the n^{th} percentile exhibiting prevalence of such antibody still yielding a statistically significant positive signal above background.

Alternatively, the measured biological value of the biological analyte, which is an antibody, especially an autoantibody, can be represented by the n^{th} consecutive dilution of the sample of said subject comprising an antibody, preferably and autoantibody against SELENOP still yielding a statistically significant positive signal above background, i.e., above the mean of negative samples+3 standard deviations (mean+3*SD of negative samples), thereby making technical noise a very unlikely reason for the positive autoantibody signal. Alternatively, the positive samples are identified by using the outlier criterium P75+1.5-times the interquartile range (P75+1.5*IQR), again making technical noise a very unlikely reason for the positive autoantibody signal. An example of these thresholds for defining positive autoantibody signals is provided in the literature, where both mean+3*SD and P75+1.5*IQR are compared [41, 42].

In one embodiment of instant invention the term “SELENOP” means human SELENOP (SELENOP), e.g., encoded by GenBank database entry CAA77836.2, respectively, according to SwissProt database number P49908.

In another embodiment of instant invention the term “SELENOP” means orthologues of SELENOP matching the respective species of the subject under investigation [43]. In further alternative embodiment of the methods of instant invention and especially with respect to the determination of the measured biological value of SELENOP, the term “SELENOP” encompasses specific fragments of human SELENOP, respectively, any orthologues thereof, which are known in the art as suitable antigens for the quantification of SELENOP. Such suitable fragments need to be specific for the quantification of SELENOP and are, known, e.g., from WO2019081504A1, especially peptide sequences according to Seq ID No.'s 3 to 15 disclosed in WO2019081504A1.

Methods for the detection of SELENOP or fragments thereof are well known to the skilled person, preferred are mass spectroscopy and immunoassay, most preferred an immunoassay. Specific SELENOP fragments are known in the art as suitable indicator molecules for the quantification of SELENOP.

In one embodiment of instant invention SELENOP is measured by means of an immunoassay using a capture molecule which binds to SELENOP and/or fragments thereof. Usually, the capture molecule is an antibody or an antibody fragment against the target molecule or antigen. However, there are numerous alternatives for capture molecules known to the skilled artisan familiar with immunoassay technologies.

Determining of SELENOP usually includes the immunoreactivity towards a region within the aforementioned molecules. This means that it is not necessary that a certain fragment is measured selectively. It is understood that a capture molecule which is used for the determination of the level of selenoprotein P (SELENOP) and/or fragments thereof binds to any fragment that comprises the region of binding of said capture molecule. Said capture molecule may be an antibody or antibody fragment or a non-IgG Scaffold. A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), chemiluminescence- and fluorescence-immunoassays, Luminex-based bead arrays, protein microarray assays, and rapid test formats such as for instance immunochroma-tographic strip tests (“dipstick immunoassays”) and immuno-chromatography assays.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems:

Roche Elecsys®, Abbott Architect®, Siemens Centauer®, Brahms Kryptor®, Biomerieux Vidas®, Immunodiagnostic Systems IDS-iSYS®, Alere Triage®.

Another embodiment of the method of instant invention includes the so-called POC-test (point-of-care), which is a test technology allowing the performance of the test within less than about one hour without the requirement of a fully automated assay system. Examples for this technology may be based on the lateral flow technology, immunochromatographic, bioluminescence or fluorescence resonance energy transfer, surface plasmon resonance, biosensor or electrochemical tests.

In one embodiment of the invention at least one of said two capture molecules is labeled in order to be detectable. In a preferred embodiment said label is selected from the group comprising chemiluminescent label, enzyme label, fluorescence label, or any other label known in the art. Most preferred embodiments of each of the objects of instant invention are without radiolabeling, especially in the detection methods of and used according to instant invention or in the kits of instant invention, there is no radiolabeling present or used.

Regarding the assay architecture it is to be noted that the assays of the invention can be homogenous or heterogeneous and further that the assays of the invention can be designed competitive or non-competitive. In one embodiment of instant invention, the assay is in the form of a sandwich assay, which is a non-competitive immunoassay, wherein the molecule to be detected and/or quantified is bound to a first antibody and to a second antibody. The first antibody may be bound to a solid phase, e.g., a bead, a surface of a well or other container, a chip or strip, whereby the second antibody is an antibody which is labeled, e.g., with a dye, with a radioisotope, with a tag sequence or a reactive or catalytically active moiety. The amount of labeled antibody bound to the analyte is then measured by an appropriate method. The general composition and procedures involved with “sandwich assays” are well-established and known to the skilled person [44-46]. In an alternative embodiment of instant invention, the assay is in the form of a sandwich assay, which is a competitive immunoassay, wherein the molecule to be detected and/or quantified is bound to a first antibody and to a second antibody and wherein such binding is further competed by another binding molecule, which is preferably an element of the test kit of the invention and is sufficiently characterized to allow the absolute or relative determination of the molecule to be detected. Accordingly, such other binding molecule suitable according to instant invention can be, e.g., labelled or detectable protein fragments, peptides, antibodies, aptamers, or other specifically binding molecules. For the understanding of instant invention, the terms ‘binding’ and ‘capturing’ (or ‘binding molecule’ and ‘capture molecule’) are used as synonyms.

In other embodiments of instant invention the detection of the analyte—where applicable—may comprise two capture molecules, preferably antibodies, which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample. Said labeling system may further comprise rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, fluorescein, fluoresceinisothiocyanate (FITC), IRD-700/800, Cyanine dyes, such as CY3, CY5, CY3.5, CY5.5, Cy7, xanthen, 6-Carboxy-2′,4′,7′,4,7-hexachlorofluorescein (HEX), TET, 6-Carboxy-4′,5′-dichloro-2′,7′-dimethodyfluorescein (JOE), N, N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), rhodamine, rhodamine Green, rhodamine Red, rhodamine 110, BODIPY dyes, such as BODIPY TMR, Oregon Green, coumarines such as umbelliferone, benzimides, such as Hoechst 33258; Phenanthridines, such as Texas Red, Yakima Yellow, Alexa Fluor, PET, ethidiumbromide, acridinium dyes, carbazol dyes, phenoxazine dyes, porphyrine dyes, polymethine dyes, and the like.

In the context of the present invention, chemiluminescence based assays comprise the use of dyes, based on the physical principles described for chemiluminescent materials as known to the skilled person (49). Chemiluminescent label may be acridinium ester label, steroid labels involving isoluminol labels and the like. Preferred chemiluminescent dyes are acridiniumesters.

Examples of suitable enzyme labels are lactate dehydrogenase (LDH), creatine kinase (CPK), alkaline phosphatase (AP), secreted alkaline phosphatase (SEAP), aspartate aminotransferase (AST), alanine aminotransferase (ALT), acid phosphatase, glucose-6-phosphate dehydrogenase, luciferase (LUC), green fluorescence protein (GFP).

In one embodiment of instant invention the assays for determining SELENOP in a sample exhibit an assay sensitivity of <0.100 mg/L, preferably <0.05 mg/L and more preferably <0.01 mg/L.

According to instant invention, an “assay” or “diagnostic assay” can be of any type applied in the field of diagnostics. Preferably, assays of the invention are performed in-vitro. Such an assay may be based on the binding of an analyte to be detected to one or more capture probes with a certain affinity. Concerning the interaction between capture molecules and target molecules or molecules of interest, the affinity constant is greater than 10⁷ M⁻¹, preferred 10⁸ M⁻¹, more preferred greater than 10⁹ M⁻¹, most preferred greater than 10¹⁰ M⁻¹. Binding affinity parameters may be determined using the Biacore method, offered as service, e.g., at Biaffin, Kassel, Germany (http://www.biaffin.com/de/). Binding affinity may be determined under suitable standard test conditions, which are defined by the skilled person in the art.

In the context of the present invention, the term “capture molecule” means a molecule which may be used to bind target molecules or molecules of interest, i.e., analytes (i.e., in the context of the present invention Selenoprotein P and fragments thereof), from a sample. “Capture molecules” must thus be shaped adequately, both spatially and in terms of surface features, such as surface charge, hydrophobicity, hydrophilicity, presence or absence of lewis donors and/or acceptors, to specifically bind the target molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules of interest. “Capture molecules” may be selected from the group comprising a nucleic acid molecule, a carbohydrate molecule, a PNA molecule, a protein, an antibody, a peptide or a glycoprotein. Preferably, the “capture molecules” are antibodies, including fragments thereof with sufficient affinity (i.e., to allow a quantification of the target molecule according to the assay) to a target molecule or molecule of interest, and including recombinant antibodies or recombinant antibody fragments, as well as chemically and/or biochemically modified derivatives of said antibodies or fragments derived from the variant chain with a length of at least 12 amino acids thereof. In one embodiment of the invention at least one of said two capture molecules is bound to a solid phase as magnetic particles, or carried on a paper or polystyrene surface.

The term “Se” according to instant invention preferably means—if not otherwise indicated—the chemical element with the symbol Se and atomic number 34 and includes all naturally occuring isotopes of Se.

Methods for the detection of Se are well known to the skilled person in the art. Usually, the determination and quantification of Se is performed using atomic absorption spectroscopy, mass spectrometry (e.g., inductively coupled plasma mass spectrometry), or total reflection X-ray fluorescence. E.g., methods for the detection of Se requiring minimal sample preparation exhibiting a detection limit of 0.2 μmol/L or less for serum Se.

The term “antibody” according to instant invention preferably means any antibody or antibody analogue or derivative known by the skilled person, which is selected from antibodies e.g., IgG, a typical full-length immunoglobulin, enzymatically derived antibody fragments (e.g., Fab′, F(ab′)2, Fc), and genetically engineered antibody fragments (e.g., scFv, (scFv)2, minibody, diabody, triabody, tetrabody). Such antibody fragments may contain at least the F-variable domain of heavy and/or light chain, e.g., chemically coupled antibodies (fragment antigen binding) including but not limited to Fab-fragments including Fab minibodies, single chain Fab antibody, monovalent Fab antibody with epitope tags, e.g., Fab-V5Sx2; bivalent Fab (mini-antibody) dimerized with the C¾ domain; bivalent Fab or multivalent Fab, e.g., generated via multimerization using a heterologous domain, e.g., via dimerization of dHLX domains, e.g., Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g., from a different class than IgG; single-domain antibodies, e.g., nanobodies derived from camelid or fish immunoglobulines.

In a specific embodiment of the invention the term “antibody” comprises antibody fragments, aptamers, non-Ig scaffolds.

Furthermore, the term “antibody” according to instant invention may encompass biopolymer scaffolds which are well known to the skilled person to complex an antigen molecule with a binding affinity like an antibody, i.e., according to the invention against SELENOP, and have been used for the generation of highly antigen (SELENOP) specific biopolymers. Examples of biopolymer scaffolds are aptamers, spiegelmers, anticalins and conotoxins. Non-Ig (non-immunoglobuline) scaffolds may be protein scaffolds and may be suitable as antibody mimics in their capability to specifically bind to ligands or target molecules. Non-Ig scaffolds may be selected from the group comprising tetranectin-based non-Ig scaffolds (e.g., US20100028995A1), fibronectin scaffolds (e.g., EP1266025A1, lipocalin-based scaffolds (e.g., WO2011154420A1), ubiquitin scaffolds (e.g., WO2011073214A1). transferring scaffolds (e.g., US20040023334A1), protein A scaffolds (e.g., EP2231860A1), ankyrin repeat based scaffolds (e.g., WO2010060748A1), microproteins preferably microproteins forming a cystine knot) scaffolds (e.g., EP2314308A1), Fyn S¾ domain-based scaffolds (e.g., WO2011023685A1), EGFR-A domain-based scaffolds (e.g., WO2005040229A1) and Kunitz domain-based scaffolds (e.g., EP1941867A1)).

In one embodiment of instant invention the term “antibody” preferably means one or more autoantibodies, especially autoantibodies interacting in a subject in any disease or disorder related to a selenium deficit as defined above.

In another embodiment of instant invention the term “antibody” preferably means one or more therapeutic antibodies, especially suitable in the treatment of a subject suffering from any disease or disorder related to a selenium deficit.

Methods for the detection of antibodies against SELENOP (“Ab(SELENOP)”) are well known to the skilled person (34).

Yet another object of the present invention is a test kit for performing the method of this invention comprising
a) a specific reagent for the determination of antibodies against SELENOP (SELENOP-Ab), preferably SELENOP-aAb;
b) a user leaflet;
c) optionally one or more reagents or chemicals selected from buffer, calibration standard, negative control, positive control; and
d) optionally a specific reagent for the determination of SELENOP.

Preferably, the test kit according to instant invention is storage stable, i.e., the stability of all reagents provided in the kit is at least 6 months, preferably at least 1 year. Optionally, one or more of the reagents and/or chemicals of the test kit of the present invention is provided in a freeze-dried form.

The term “binding index of Ab(SELENOP)” (hereinafter “Ab(SELENOP)-BI”) is the relevant relative parameter of interest if an absolute determination of the amount or concentration of Ab(SELENOP) is not possible, e.g., as in most cases of autoantibody detection.

The expression “evaluating the risk for selenium deficiency in a subject” according to instant invention preferably means the identification of the selenium status of a subject by use of the parameters as taught by instant invention, i.e., Se and/or SELENOP together with Ab(SELENOP), and optionally correlating the identified selenium status of the subject with its need or requirement of a Se supplementation for prophylactic (e.g., in conditions of increased exposure to toxicants, virus or bacteria) preventive (e.g., for cancer, cardiovascular or autoimmune disease risk reduction), or therapeutic purposes (e.g., as an adjuvant therapy during treatment of infection, subfertility, in cancer, autoimmune or cardio-vascular diseases).

The term “Se supplementation” according to instant invention, whether used in a preventive or prophylactic, therapeutic or nutritional (i.e., as adjuvant or sole treatment) context, preferably means a Se intake by a subject in a pharmaceutically acceptable amount. Se may be applied in the form of physiological acceptable selenite, selenate, selenomethionine (L-selenomethionine), or selenium-rich food products, whether biofortified by Se or not (e.g., brazil nut, vegetables, yeast, fruits). In preferred embodiments of the methods of the invention the Se supplementation or Se therapy is performed by the administration of Se to the subject in need thereof, which is selected from selenite, selenate or selenomethionine (L-selenomethionine), optionally in combination with an anti-oxidant, especially co-enzyme Q10, glutathione, vitamin C or E, zinc or other micronutrients.

If a significant Se deficit has been detected, Se supplementation may exceed the tolerable upper intake limit (UL(Se)) according to the present knowledge of the skilled person in the art or exceeds the UL(Se), provided the Se status of the subject is monitored. Optionally, a Se supplementation may be applied in combination with vitamin products or vitamins (e.g., vitamin E, vitamin C, vitamin A) and/or mineral nutrients (e.g., iodine, fluoride, zinc, copper, iron) and/or co-factors (e.g., coenzyme Q10). Dosed formulations for Se supplementation are available as, e.g., tablets, capsules, granules, powders, sachets, in water soluble powders, liquids or fluids for peroral use or for intravenous administration to the subject in need thereof.

Another embodiment of instant invention is the use of the methods of the invention for evaluating the risk of selenium (Se) deficiency in a subject for companion diagnostics or monitoring in the quantification of the required individual Se amount and dosage regime during Se supplementation therapy in a subject.

According to instant invention the term “risk index score for Se deficiency” hereinafter “RIS(Se)” means the parameter for improved definition and classification of the Se status of an individual. In one embodiment of instant invention the RIS(Se) is calculated according to Formula I:

$$RIS ( Se ) = Mark ( Se equivalent ) + Mark ( Ab ( SELENOP ) - BI ) \qquad ( Formula I )$$

Thus, the RIS(Se) Scoring may result from addition of the mark values in accordance determined in a sample of a subject according to the methods of instant invention, whereby in one preferred embodiment of instant invention all measured biological values for this calculation result from one sample of said subject.

TABLE 1A
Pre-Scoring of Mark distribution for the determined mark
of the measured biological values of the amount of Se
equivalent (Se or SELENOP, *serum or plasma level)

| | | |
|---|---|---|
| Mark | Amount Se* resp. | Amount SELENOP* |

| 1 | >120 | μg/L | >6 | mg/L |
|---|---|---|---|---|
| 2 | >90 to 120 μg/L | | >4 to 6 mg/L | |
| 3 | >70 to 90 μg/L | | >3 to 4 mg/L | |
| 4 | > or equal 35 to 70 μg/L | | > or equal 1.5 to 3 mg/L | |
| 5 | <35 | μg/L | <1.5 | mg/L |

TABLE 1B
Pre-Scoring of Mark distribution for the determined mark
of the measured biological values of the binding index
of antibodies against SELENOP (Ab(SELENOP)-BI)

| Mark | Binding Index Ab(SELENOP) |
|---|---|
| 1 | 1 to 3 |
| 2 | >3 to 5 |
| 3 | >5 to 7.5 |
| 4 | >7.5 to 10 |
| 5 | >10 |

TABLE 2
RIS(Se) Scoring using Formula I, classification of Selenium
status and requirement for Se supplementation

| Score | Conclusion regarding Se supplementation |
|---|---|
| 1 to <3 | means no Se deficit, no Se supplementation required |
| 3 to <4 | means a mild Se deficit, mild Se suppl. can be considered |
| 4 to 5 | means a moderate Se deficit, moderate Se suppl. required |
| >5 | means a severe Se deficit, high Se supplementation required |

If both biological parameters (Se and SELENOP) of a sample have been determined, the average value or a weighted average value (e.g., corresponding to the statistical error bars) may be generated and used in Formula I.

It is well within the knowledge of the skilled person that it is possible to allocate different Mark values for the used parameters, which will result in different score ratings as an immediate result of the calculation. It is also well within the knowledge of the skilled person that it is possible to use one or more other mathematical operators in Formula I for calculating the named parameters, which will result in the same or different score rating as an immediate result of the calculation. It is also well within the knowledge of the skilled person that it is possible to use both, different Mark values for the used parameters, and one or more other mathematical operators for calculating the named parameters, which will result in the same or different score ratings as an immediate result of the calculation. Suitable mathematical operators for the calculation of a RIS scoring according to instant invention are, e.g., multiplication, division, addition, subtraction, potentiation of the measured values (i.e., the marks or raw data values) by a number, and others. This means that an alternative formula 1′ may read, e.g., RIS(Se)=Mark(Se amount)\hat{ }^{x} [operator] Mark(SELENOP)\hat{ }^{y} [operator] Mark(Ab(SELENOP)-BI)\hat{ }^{z}, wherein x, y and z are selected from real numbers.

It is explicitly mentioned that alternative approaches for the calculation of the RIS(Se) may be valid and are within the gist of instant invention as far as a measured value for Ab(SELENOP) is introduced into such calculation in a manner that an increasing amount of Ab(SELENOP) generally correlates with an increasing risk of Se deficiency, respectively an increasing recommendation for Se supplementation.

Further embodiments of the present invention are also methods comprising the supplementation with Se in subjects identified to be at high risk by use of the methods for evaluating the risk of selenium (Se) deficiency in a subject according to instant invention, especially of a subject exhibiting a RIS(Se) Scoring as outlined herein in Formula 1 of 3 or more.

Another embodiment of instant invention is a method of treating a subject suffering from one or more DRSD with Se supplementation, wherein any of the methods for evaluating the risk of selenium (Se) deficiency according to the invention is performed and used as companion diagnostics, wherein the term “companion diagnostics” means the monitoring of the Se status, preferably wherein the calculation of the RIS(Se) as defined by instant invention is used, more preferred wherein the subject is suffering from one or more DRSD, in particular i) sepsis, ii) inflammation, iii) SIRS, iv) inflammatory bowel diseases, v) infection, vi) cardio-vascular diseases (CVD), vii) atherosclerosis, viii) cardiovascular event, ix) stroke, x) myocardial infarction, xi) cardiomyopathy, xii) cancer, xiii) breast cancer, xiv) prostate cancer, xv) colorectal cancer, xvi) lung cancer, xvii) liver cancer, xviii) (poly)trauma, xix) thyroid dysfunction, xx) diabetes mellitus type I and/or II, xxi) a severe illness treated in an intensive care unit (ICU), xxii) severe burn or scald injury, xxiii) poisoning, xxiv) a neurodegenerative disease, xxv) epileptic seizure, xxvi) Alzheimer's disease, xxvii) Parkinson's disease, xxviii) Huntington's disease, xxix) progressive or chronical multiple sclerosis (MS), xxx) amyotrophic lateral sclerosis (ALS), xxxi) infertility, or xxxii) hypertension, and in specific embodiments Hashimoto's disease, Graves' ophtalmopathy, influenza A, hepatitis B, hepatitis C, or COVID-19.

Another embodiment of instant invention is a method of treating a subject suffering from a disease related to a selenium deficit with Se supplementation, wherein any of the methods for evaluating the risk of selenium (Se) deficiency according to the invention is performed and used for therapeutical guidance, preferably wherein the calculation of the RIS(Se) as defined by instant invention is used, more preferred wherein the subject is suffering from one or more DRSD, in particular i) sepsis, ii) inflammation, iii) SIRS, iv) inflammatory bowel diseases, v) infection, vi) cardio-vascular diseases (CVD), vii) atherosclerosis, viii) cardiovascular event, ix) stroke, x) myocardial infarction, xi) cardiomyopathy, xii) cancer, xiii) breast cancer, xiv) prostate cancer, xv) colorectal cancer, xvi) lung cancer, xvii) liver cancer, xviii) (poly)trauma, xix) thyroid dysfunction, xx) diabetes mellitus type I and/or II, xxi) a severe illness treated in an intensive care unit (ICU), xxii) severe burn or scald injury, xxiii) poisoning, xxiv) a neurodegenerative disease, xxv) epileptic seizure, xxvi) Alzheimer's disease, xxvii) Parkinson's disease, xxviii) Huntington's disease, xxix) progressive or chronical multiple sclerosis (MS), xxx) amyotrophic lateral sclerosis (ALS), xxxi) infertility, or xxxii) hypertension, and in specific embodiments Hashimoto's disease, Graves' ophtalmopathy, influenza A, hepatitis B, hepatitis C, or COVID-19.

Another embodiment of instant invention is a method of treating a subject suffering from a disease related to a selenium deficit with Se supplementation, wherein any of the methods for evaluating the risk of selenium (Se) deficiency according to the invention is performed at least two times, preferably wherein the calculation of the RIS(Se) as defined by instant invention is used, more preferred wherein the subject is suffering from one or more DRSD, in particular i) sepsis, ii) inflammation, iii) SIRS, iv) inflammatory bowel diseases, v) infection, vi) cardio-vascular diseases (CVD), vii) atherosclerosis, viii) cardiovascular event, ix) stroke, x) myocardial infarction, xi) cardiomyopathy, xii) cancer, xiii) breast cancer, xiv) prostate cancer, xv) colorectal cancer, xvi) lung cancer, xvii) liver cancer, xviii) (poly)trauma, xix) thyroid dysfunction, xx) diabetes mellitus type I and/or II, xxi) a severe illness treated in an intensive care unit (ICU), xxii) severe burn or scald injury, xxiii) poisoning, xxiv) a neurodegenerative disease, xxv) epileptic seizure, xxvi) Alzheimer's disease, xxvii) Parkinson's disease, xxviii) Huntington's disease, xxix) progressive or chronical multiple sclerosis (MS), xxx) amyotrophic lateral sclerosis (ALS), xxxi) infertility, or xxxii) hypertension, and in specific embodiments Hashimoto's disease, Graves' ophtalmopathy, influenza A, hepatitis B, hepatitis C, or COVID-19.

Yet another object of instant invention is a method for the quantification of the individual Se requirement and dosage regime during therapeutic or prophylactic Se supplementation in a subject by using the method for evaluating the health risk of Se deficiency according to instant invention or the risk index score obtainable according to instant invention.

Still another object of instant invention is a method of monitoring Se supplementation in a subject as a companion diagnostic by using the method for evaluating the health risk of Se deficiency according to instant invention or the risk index score obtainable according to instant invention. Yet another object of instant invention is a method of monitoring the treatment of a subject suffering from selenosis by administration of a pharmaceutically effective amount of an antibody against SELENOP as a companion diagnostic by using the method for evaluating the health risk of Se deficiency according to instant invention or the risk index score obtainable according to instant disclosure.

Yet another object of instant invention is a method for the analysis and monitoring of Se poisoning by using the method for evaluating the health risk of Se deficiency according to instant invention or the risk index score obtainable according to instant invention.

Still another object of instant invention is a method of monitoring the treatment of a subject suffering from a disease selected from sepsis, infection, (poly)trauma or SIRS by using the method for evaluating the health risk of Se deficiency according to instant invention or the risk index score obtainable according to instant invention.

Yet another object of instant invention is a method of monitoring the treatment of a subject suffering from a viral infection selected from influenza A and COVID-19 by using the method for evaluating the health risk of Se deficiency according to instant invention or the risk index score obtainable according to instant invention.

Still other objects of instant invention are a) the use of a pharmaceutically effective amount of an antibody against SELENOP in the treatment of a subject suffering from selenosis; b) the manufacturing of a pharmaceutical composition for the treatment of a subject suffering from selenosis comprising a pharmaceutically effective amount of an antibody against SELENOP and optionally further auxiliary ingredients; and c) a pharmaceutical composition for the treatment of a subject suffering from selenosis comprising a pharmaceutically effective amount of an antibody against SELENOP. Such antibodies against SELENOP are known in the art [50]. The treatment of a subject by using a pharmaceutically effective amount of an antibody against SELENOP is preferably accompanied by monitoring the SE status by use of the methods for evaluating the risk of selenium (Se) deficiency according to instant disclosure.

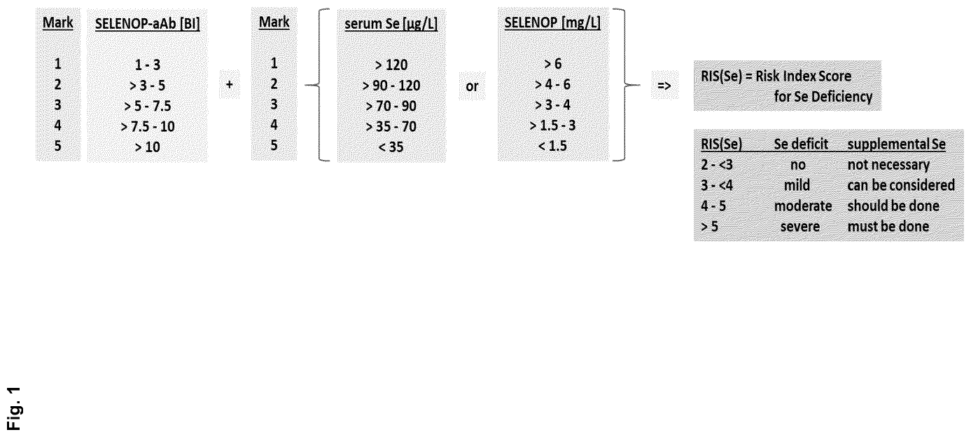

FIG. 1 illustrates the scheme for applying the Risk Index Score for Se deficiency in accordance with the relevant applicable threshold values.

### SEQUENCES

SEQ ID No 1: Primer P1
SEQ ID No 2: Primer P2
SEQ ID No 3: cDNA of recombinant SEAP
SEQ ID No 4: Primer P3
SEQ ID No 5: Primer P4
SEQ ID No 6: cDNA of human recombinant SELENOP
SEQ ID No 7: cDNA of fusion protein SELENOP-SEAP
SEQ ID No 8: amino acid sequence of fusion protein SELENOP-SEAP

### EXAMPLES

### Materials:

DNA primer were obtained from Life Technologies (Carlsbad, CA, USA), vectors from Promega GmbH (Mannheim, Germany), pIRESneo vector from Clontech (Palo Alto, CA, USA). If not otherwise specified, other chemicals and reagents were obtained from Sigma-Aldrich Chemie GmbH (Munich, Germany) or Merck KGAA (Darmstadt, Germany), enzymes from Promega (Madison, WI, USA) or New England Biolabs (Ipswich, MA, USA). Cell culture media and additives were obtained from Fisher Scientific Co. LLC, (Hanover Park, Ill., USA). Percentage values in the context of liquids are given in (v/v), otherwise the values are (w/w). Room temperature (RT) means 20° C.+/−5° C.

### Example 1: Determination of Selenoprotein P in Serum Samples

The selenOtest™ ELISA is a chromogenic enzyme-linked immunosorbent assay, for the quantitative determination of human SELENOP in serum samples. It uses two different SELENOP specific monoclonal antibodies for the antigen capture and detection steps, essentially as described [7]. SELENOP concentration in IgG-isolates or serum samples were measured by sandwich ELISA using a validated commercial SELENOP-specific ELISA with two monoclonal antibodies (mAb1 and mAb2). The SELENOP concentrations of the calibrators and controls have been validated against serial dilutions of NIST SRM 1950 Standard Reference Material (National Institute of Standards & Technology, Gaithersburg, Maryland, USA). The lower limit of quantification (LLOQ) was determined at a SELENOP concentration of 11.6 μg/L, and the upper limit of quantification (UL(Se)OQ) at 538.4 μg/L, thereby defining the working range in diluted serum samples at SELENOP concentrations between 11.6 and 538.4 μg/L. The intersection at 20% CV defines the limit of detection (LOD) and was reached at a SELENOP concentration of 6.7 μg/L, i.e., around 500-fold below average serum SELENOP concentrations of well-supplied human subjects. The signals were linear on dilution within the working range of the assay, and SELENOP was stable in serum for 24 h at room temperature. Further details of the assay parameters are published [7], and the assay has been proven as a most reliable product in comparison to other competing products [47].

Briefly, each 100 μL of positive and negative IgG-isolates without ethanol-precipitation were applied to pre-coated 96-well plates. Serum samples (5 μL) were diluted 1:33 and applied to pre-coated 96-well plates. Standards and calibrators (positive and negative controls) were included into each assay run.

### Example 2: Determination of Selenium in Serum Samples

Selenium concentrations in the IgG-isolates or serum samples were analyzed by total reflection X-ray fluorescence (TXRF) using a benchtop TXRF analyser (picofox S2, Bruker Nano GmbH, Berlin, Germany) as generally described (48). To this end, the proteins from SELENOP-aAb positive and SELNOP-aAb negative IgG isolates were precipitated using 9-fold volume of 100% ice-cold ethanol to 1-fold volume IgG isolate, and incubated overnight at −20° C. After centrifugation for 30 min at 15000*g, 4° C., the supernatants were discarded, and pellets were incubated at 80° C. for 2 h in 100 μL 61% HNO₃ with a Ga-Standard concentration of 1 mg/L. The serum samples were directly 1:2 diluted with a Ga-Standard concentration of 1 mg/L. For Se-detection by TXRF, 8 μL of each solution was applied to a polished quartz glass slide and dried overnight.

### Example 3: Determination of Antibodies Against SELENOP in Serum Samples

### 3.1 Generation of Recombinant SEAP-SELENOP Reporter Proteins

Construction of pIRESneo-SEAP Plasmid

The cDNA sequence of secreted alkaline phosphatase (SEAP) (pSEAP2-Basic, Clontech) encoding amino acids 1-513 (SEQ ID No.3) was amplified by PCR using primer P1 (5′-3′: atagatatcatgctgctgctgctgctgctg, SEQ ID No.1) and primer P2 (5′-3′: atagcggccgccccgactctagagtaacccgg, SEQ ID No.2) containing EcoRV and NotI restriction sites. pIRESneo plasmid (Clontech, Palo Alto, California) was digested with EcoRV and NotI restriction endonucleases (NEB Bioloabs), the fragment was removed and replaced with the PCR sequence giving rise to pIRESneo-SEAP plasmid.

The cDNA of human recombinant SELENOP (SEQ ID No. 6) was amplified by PCR using primers P1 (5′-3′: atagcggccgctgagagccaggaccaaagctcctta) (SEQ ID No. 4) and P2 (5′-3′: atagaattcttagtttgaagggcattcgcactt) (SEQ ID No. 5) (BioTeZ, Berlin, Germany). Both sequences were extended providing suitable restriction sites. After restriction, digestion, and isolation via agarose gel electrophoresis, the isolated DNA encoding the restricted SELENOP cDNA was ligated into vector pIRESneo-SEAP and subsequently transformed into E. coli. Positive clones of the SEAP-SELENOP coding sequence (SEQ ID No. 7) were identified and the expression plasmids were verified by sequencing. For the protein production of the fusion protein SEAP-SELENOP (SEQ ID No. 8), human embryonic kidney cells (HEK 293 cells) were cultured in DMEM/F12 supplemented with 10% fetal bovine at 37° C. and 5% CO2 and transfected with PIRESneo-SEAP-SELENOP using FuGENER reagent (Promega). 48 h after transfection, successfully transfected cells were selected by cultivating in DMEM/F12 with 10% (v/v) FBS and 0.5-0.8 mg/ml G418. Stable clones expressing high levels of recombinant protein (SEAP-SELENOP) were expanded and the cell supernatants were collected.

### 3.2 Immunoluminometric Assay for the Detection of aAb Against SELENOP

An immunoluminometric assay for detection of autoantibodies (“aAb”) to SELENOP (“SELENOP-aAb”) was established and used to analyse serum samples. The immunoluminometric assay is based on the binding of aAb to the recombinant SELENOP fused to the reporter SEAP, followed by precipitation of this antibody-antigen-reporter complex by protein A. To this end, each 40 μL of 1:100 diluted antigen-reporter supernatants were incubated with 10 μL of 1:2 diluted serum sample at 4° C. overnight. On the second day, 40 μl protein A slurry (20%) was added, incubated for 1 h at 25° C., washed (50 mM Tris-HCl, pH 7.5, 100 mM NaCl, 0.5% Triton X-100) and centrifuged at 500*g (4° C., 5 min.). After six washing steps, the reporter activity was detected as a relative luminescence signal (RLU), which is proportional to the amount of SELENOP-aAb in the sample.

### 4. Statistical Analysis

The results are represented as means±SD or distribution of the individual values. Data in SELENOP-aAb assays were evaluated for each assay-plate. Mean value of the 50% smallest signal intensity in a same plate was set to 1 relative unit (rel unit, RU, resp., rel. luminescence units, RLU). The relative SELENOP aAb titer in the sample was calculated as quotient of individual signal intensity and the mean value of the 50% smallest signal intensity in the same plate, based on the assumption that SELENOP-aAb is not present in more than 50% of the population. A serum sample was defined as positive of SELENOP-aAb, if the calculated quotient of the signals was higher than five or ten times the average background signal of negative (control) samples, yielding a cut-off=5 or cut-off=10. The inter- and intra-assay CV was determined to be below 20%.

### REFERENCES

1. Burk, R. F. and K. E. Hill. Annu Rev Nutr, 2015. 35: p. 109-34.
2. Hurst, R., et al. Am J Clin Nutr, 2010. 91(4): p. 923-31.
3. Combs, G. F., Jr., et al. Br J Nutr, 2012. 107(10): p. 1514-25.
4. Ashton, K., et al. American J of Clinical Nutrition, 2009. 89(6): p. 2025s-2039s.
5. Xia, Y. M., et al. American J of Clinical Nutrition, 2010. 92(3): p. 525-31.
6. Kipp, A. P., et al. J Trace Elem Med Biol, 2015. 32: p. 195-9.
7. Hybsier, S., et al. Redox Biology, 2017. 11: p. 403-14.
8. Burk, R. F. and K. E. Hill. Annu Rev Nutr, 2005. 25: p. 215-35.
9. Saito, Y. Journal of Clinical Biochemistry and Nutrition, 2020. 66(1): p. 1-7.
10. Saito, Y., et al. Biochemical Journal, 2004. 381: p. 841-6.
11. Ballihaut, G., et al. Metallomics, 2012. 4(6): p. 533-8.
12. Hill, K. E., et al. Journal of Nutrition, 1996. 126(1): p. 138-45.
13. Persson-Moschos, M., et al. Eur J Clin Nutr, 1998. 52(5): p. 363-7.
14. Xia, Y. M., et al. American J of Clinical Nutrition, 2005. 81(4): p. 829-34.
15. Burk, R. F., et al. Cancer Epidemiol Biomarkers Prev, 2006. 15(4): p. 804-10.
16. Rayman, M. P., et al. Br J Nutr, 2014. 112(1): p. 99-111.
17. Kahaly, G. J., et al. J Clin Endocrinol Metab, 2017. 102(11): p. 4333-41.
18. Chen, M. X., et al. Obesity Research & Clinical Practice, 2017. 11(2): p. 227-32.
19. Kikuchi, N., et al. Circulation, 2018. 138(6): p. 600-23.
20. Yang, S. J., et al. J Clin Endocrinol Metab, 2011. 96(8): p. E1325-9.
21. Hollenbach, B., et al. J Trace Elem Med Biol, 2008. 22(1): p. 24-32.
22. Renko, K., et al. FASEB J, 2009. 23(6): p. 1758-65.
23. Gharipour, M., et al. J Gene Med, 2017. 19(3) p. e2945.
24. Senthilkumaran, S., et al. Int J Trichology, 2012. 4(4): p. 283-4.
25. Morris, J. S. and S. B. Crane. Nutrients, 2013. 5(4): p. 1024-57.
26. Hill, K. E., et al. J Nutr, 1996. 126(1): p. 138-45.
27. Xia, Y., et al. American J Clin Nutr, 2010. 92(3): p. 525-31.
28. Eskes, S. A., et al. Clinical Endocrinology, 2014. 80(3): p. 444-51.
29. Yang, J. G., et al. J Nutr, 1989. 119(7): p. 1010-2.
30. Renko, K., et al. Biochem J, 2008. 409(3): p. 741-9.
31. Moghaddam, A., et al. Nutrients, 2020. 12(7):2098.
32. Schomburg, L. Hormones (Athens), 2020. 19(1): p. 15-24.
33. Schomburg, L. Nat Rev Endocrinol, 2011. 8(3): p. 160-71.
34. Sun, Q., et al., Autoimmunity to Selenoprotein P in Thyroid Patients. 62. Kongress für Endokrinologie, Göttingen, Germany, 2019.
35. Persson-Moschos, M., et al. Analyst, 1995. 120(3): p. 833-6.
36. Huang, W., et al. British Journal of Nutrition, 1995. 73(3): p. 455-61.
37. Andoh, A., et al. Nutrition, 2005. 21(5): p. 574-9.
38. Hybsier, S., et al. Redox Biol, 2017. 11: p. 403-14.
39. Perssonmoschos, M., et al. Analyst, 1995. 120(3): p. 833-36.
40. Tanaka, M., et al. J Clin Lab Anal, 2016. 30(2): p. 114-22.
41. Kerman, R., et al. Clin Transpl, 2013: p. 357-60.
42. Schwiebert, C., et al. Int J Mol Sci, 2020. 21(2):463
43. Penglase, S., et al. PeerJ, 2015. 3: p. e1244.
44. Poschenrieder, A., et al. Anal Bioanal Chem, 2019. 411(29): p. 7607-21.
45. Justino, C. I., et al. Adv Clin Chem, 2016. 73: p. 65-108.
46. Del Campo, M., et al. Front Neurol, 2015. 6: p. 202.
47. Saito, Y., et al. Biological & Pharmaceutical Bulletin, 2018. 41(5): p. 828-33.
48. Hoeflich, J., et al., (2010) Br J Nutr 104(11): 1601-1604.
49. Kirk-Othmer, Encyclopedia of chemical technology, 4th ed., executive editor, J I Kroschwitz; editor, M Howe-Grant, John Wiley & Sons (1993) Vol. 15, pp. 518-562.
50. Mita, Y., et al., (2017). Nat. Commun. 8: p. 1658.

1. A method for evaluating the health risk of selenium (Se) deficiency in a subject comprising:
a) determining in a sample of said subject a measured biological value selected from
a1) selenium (Se) and/or a2) selenoprotein P (SELENOP); and
b) determining in a sample of said subject a measured biological value of antibodies against SELENOP (SELENOP-Ab).

2. The method for evaluating the health risk of selenium (Se) deficiency in a subject according to claim 1 further comprising the steps of:
c) setting two or more biological parameters into correlation, wherein said biological parameters are represented by the said measured biological values obtainable as defined in steps a) and b) of claim 1; and
d) calculating a risk index score for the Se deficiency of said subject using the values obtainable as defined i) in step a) and b) of claim 1 or ii) in step c).

3. The method according to claim 1, wherein the said measured biological values are correlated with the risk related to Selenium deficiency in said subject, wherein said subject is suffering from a disorder selected from sepsis, inflammation, SIRS, inflammatory bowel diseases, infection, cardio-vascular diseases (CVD), atherosclerosis, cardiovascular event, stroke, myocardial infarction, cardiomyopathy, cancer, breast cancer, prostate cancer, colorectal cancer, lung cancer, liver cancer, (poly)trauma, thyroid dysfunction, diabetes mellitus type I and/or II, a severe illness treated in an intensive care unit (ICU), severe burn or scald injury, poisoning, a neurodegenerative disease, epilepsia, Alzheimer's disease, Parkinson's disease, Huntington's disease, progressive or chronical multiple sclerosis (MS), amyotrophic lateral sclerosis (ALS), infertility, or hypertension.

4. The method according to claim 1, wherein step a2 and/or step b is performed as an immunoassay.

5. The method according to claim 1, which includes a spectrometric method selected from mass spectrometry, fluorescence spectrometry, X-ray fluorescence spectroscopy, and absorption spectrometry.

6. The method of using the method as defined in claim 1 as a companion diagnostic method to monitor Se supplementation in a subject.

7. The method of using the method as defined in claim 1 in the quantification of the individual Se requirement and dosage regime during therapeutic or prophylactic Se supplementation in a subject.

8. The method of using the method as defined in claim 1 in the analysis and monitoring of Se poisoning or the treatment of a subject suffering from selenosis by administration of a pharmaceutically effective amount of an antibody against SELENOP.

9. The method of using the method as defined in claim 1 in the treatment of a subject suffering from a disease selected from sepsis, infection, (poly)trauma or SIRS.

10. The method of using the method as defined in claim 1 in the treatment of a subject suffering from a disease selected from viral infection including influenza A and COVID-19.

11. A kit for performing the method as defined in claim 1 comprising
a) a specific reagent for the determination of antibodies against SELENOP (SELENOP-Ab),
b) a user leaflet;
c) optionally one or more reagents or chemicals selected from buffer, calibration standard, negative control, positive control; and
d) optionally a specific reagent for the determination of SELENOP.

12. The method according to claim 2, wherein the said measured biological values are correlated with the risk related to Selenium deficiency in said subject, wherein said subject is suffering from a disorder selected from sepsis, inflammation, SIRS, inflammatory bowel diseases, infection, cardio-vascular diseases (CVD), atherosclerosis, cardiovascular event, stroke, myocardial infarction, cardiomyopathy, cancer, breast cancer, prostate cancer, colorectal cancer, lung cancer, liver cancer, (poly)trauma, thyroid dysfunction, diabetes mellitus type I and/or II, a severe illness treated in an intensive care unit (ICU), severe burn or scald injury, poisoning, a neurodegenerative disease, epilepsia, Alzheimer's disease, Parkinson's disease, Huntington's disease, progressive or chronical multiple sclerosis (MS), amyotrophic lateral sclerosis (ALS), infertility, or hypertension.

13. The method according to claim 2, wherein step a2 and/or step b is performed as an immunoassay.

14. The method according to claim 2 including a spectrometric method selected from mass spectrometry, fluorescence spectrometry, X-ray fluorescence spectroscopy, and absorption spectrometry.

15. The method of using the risk index score obtainable according to claim 2 as a companion diagnostic method to monitor Se supplementation in a subject.

16. The method of using the risk index score obtainable according to claim 2 in the quantification of the individual Se requirement and dosage regime during therapeutic or prophylactic Se supplementation in a subject.

17. The method of using the risk index score obtainable according to claim 2 in the analysis and monitoring of Se poisoning or the treatment of a subject suffering from selenosis by administration of a pharmaceutically effective amount of an antibody against SELENOP.

18. The method of using the risk index score obtainable according to claim 2 in the treatment of a subject suffering from a disease selected from sepsis, infection, (poly)trauma or SIRS.

19. The method of using the risk index score obtainable according to claim 2 in the treatment of a subject suffering from a disease selected from viral infection including influenza A and COVID-19.

20. The method according to claim 3, wherein step a2 and/or step b is performed as an immunoassay.
